(19) United States
(12) Patent Application Publication
(10) Pub. No.: US 2010/0099955 A1
(43) Pub. Date: Apr. 22, 2010

(54) Method for Transmitting Information for a Collective Rendering of Information on Emotions

(75) Inventors: Henry Thomas, Rennes (FR); Sylvie Jumpertz, Rennes (FR); Delphine Guegan, Betton (FR)

Correspondence Address: WESTMAN CHAMPLIN & KELLY, P.A., SUITE 1400, 900 SECOND AVENUE SOUTH, MINNEAPOLIS, MN, 55402, US

(73) Assignee: FRANCE TELECOM, Paris (FR)

(21) Appl. No.: 12/527,747

(22) PCT Filed: Feb. 22, 2008
(86) PCT No.: PCT/FR08/50302

§ 371 Date: Dec. 9, 2009

(30) Foreign Application Priority Data
Feb. 28, 2007 (FR) 0701606

(51) Int. Cl. A61B 5/00; G06F 15/16; G06F 17/30; G06F 3/048

(52) U.S. Cl. 600/300; 709/204; 707/769; 715/764; 707/E17.014

(57) ABSTRACT

A method is provided for transmitting information concerning at least two persons. The method for transmitting information inside a group includes at least the following steps: obtaining emotional conditions associated with the persons; converting each of the emotional conditions into condition information; transmitting the condition information to emotion rendering device capable of associating with each person at least two predetermined conditions representative of at least two respective different emotional conditions.

### CROSS-REFERENCE TO RELATED APPLICATIONS

This Application is a Section 371 National Stage Application of International Application No. PCT/FR2008/050302, filed Feb. 22, 2008 and published as WO2008/113947 on Sep. 25, 2008, not in English.

### STATEMENT REGARDING FEDERALLY SPONSORED RESEARCH OR DEVELOPMENT

None.

### THE NAMES OF PARTIES TO A JOINT RESEARCH AGREEMENT

None.

### FIELD OF THE DISCLOSURE

The present disclosure is situated in the field of telecommunications and more particularly in the sphere of communicating objects connected to a network.

More specifically, the disclosure pertains to new modes of transmitting and rendering pieces of collective information on various individuals or various members of a group connected by a communications network. These pieces of information may correspond for example to information on moods, states of mind, health, degree of fatigue etc. pertaining to the persons considered. Here below, a piece of information of this kind is called an “emotional state”.

### BACKGROUND OF THE DISCLOSURE

Such emotional states are generally easy to detect intuitively, when the speakers are in the presence of one another. However, this is not the case when the speakers are at a distance or simply connected by conventional means of communications enabling the transmission of only written, vocal and/or video messages.

There are various approaches by which an individual can share information with other individuals or with all the members of a group to which he or she belongs. To know the emotional state of other persons or other members of the group, a first known technique obviously consists in trying to contact each of the persons considered one after the other to ascertain their emotional state, for example by telephone, electronic mail or an instant messaging service etc.

One drawback of this first technique naturally is that the individual must actually take action to contact each person whose emotional state he wishes to know.

Another drawback of this technique is that the individual must renew these contacts regularly. Otherwise there is a risk of remaining with emotional states that are no longer up-to-date since moods can change very quickly.

Yet another drawback of this technique is that it cannot be used simply to share information on the emotional state of each member of a group between all the members of the group. Indeed, it is for the individual to decide to disseminate this information if he wishes to.

A second prior-art technique for disseminating information between two individuals implements two matched communicating objects connected to the Internet, for example “Nabaztag®” type objects (according to the trade name chosen by the firm Violet) as described especially in page 154 of No. 1058, November 2005 of the journal “Science et Vie”. According to this technique, when a first individual places an ear of his “Nabaztag®” device in any position whatsoever, this position is reproduced remotely on the “Nabaztag®” of the second individual, enabling these two individuals to exchange information, for example on their moods, according to a coding that they have agreed upon.

One drawback of this second technique is that it is specifically dedicated to communications between two persons equipped with two apparatuses linked to each other. It cannot be used simply to render the reciprocal emotional states of a group of more than two individuals, unless it is planned to have as many “Nabaztag®” devices as there are members in the group.

Furthermore, this technique is limited to the remote reproduction of an action performed by an individual. It does not allow each user to parameterize the rendering of the information in a different form according to his needs and preferences.

### SUMMARY

An embodiment of the invention provides a solution that does not show these drawbacks by means of a method for transmitting information pertaining to at least two individuals.

According to an embodiment of the invention, this method includes at least the following steps:
obtaining emotional states associated with said persons;
transforming each of said emotional states into a piece of state information;
transmitting said pieces of state information to emotion-rendering means capable of associating, with each person, at least two rendering elements each capable of taking at least two predetermined states representing at least two distinct respective emotional states.

Thus, an embodiment of the invention can be used to render said emotional states via the rendering means as a function of said state information on emotions.

These rendering means or communicating objects are for example objects from the home context and may be, inter alia, an accessory such as a set of photograph frames for example, a computer, a personal digital assistant, a mobile telephone, and more generally any object designed to take several states representing these states of emotion, and capable of being connected to an access to a communications network (as the case may be through a terminal or a gateway) and especially a computer network of the Internet type.

This rendering can be implemented especially by means of a visual signal or again a message or a sensory reference in the form of a sound effect, a taste or tactile and/or taste effect or resulting from a command in motion of a mechanism.

Thus, an embodiment of the invention enables the transmission and rendering appreciably in real time of information on the transient moods or states of mind of people belonging for example to a group or more generally information on the emotional states of these persons at a precise point in time.

More generally, the method of an embodiment of the invention enables these persons to know the most recent emotional state or at least the most recently declared emotional state of all the other individuals.

As already stated, the term “emotional state” refers to information, such as information on moods or various states of mind such as, for example, happy, curious, tense, irritated, disillusioned, tired or no particular state of mind (also called “neutral” here below in the description).

This method also makes it possible to share, if desired, one's own emotional state with other persons. This may be done by acting on the rendering means or by means of any other terminal connected to the network.

In at least one particular embodiment of the invention, a same rendering element can be used for successively rendering the emotional states of two different persons.

According to one particular embodiment of the invention, a method for transmitting information such as this comprises a step for putting at least one second person into communication with a first person who has preliminarily declared his emotional state.

Thus, if the emotional state declared by the first person is alarming, for example signifying that he or she is angry or in despair, an embodiment of the invention can provide for a systematic procedure, in other words an automatic and non-requested procedure for calling or setting up communications between the second person and the first person to find out how he is and try and soothe him or comfort him. Such a function may for example be provided by judiciously connecting the rendering means with calling means. In one variant of this embodiment, it can be planned that the rendering means can be used to suggest to the second person that he should call the first person, for example by clicking on an avatar.

In at least one particular embodiment of the invention, the obtaining step comprises a step of declaration, by at least one of said persons, of his emotional state.

Thus, the different persons may participate in the updating of information representing their emotional states so as to make the use and/or the service provided by an embodiment of the invention dynamic.

According to one particular aspect of an embodiment of the invention, such a declaring step comprises a step for associating a date of creation with said emotional state.

Thus, the rendering means are capable of rendering firstly the emotional state and secondly the date of updating and/or the time that has elapsed since the declaring of this emotional state. The term “date” is understood here to mean not only the identification of a day but naturally more generally the declaration of an instant, if necessary with a time of declaration.

According to one particular aspect of an embodiment of the invention, the step for transforming comprises a step for adapting the state information as a function of said date of creation so as to enable a rendering of a degree of obsolescence of said state information.

Thus for example, if the rendering of the state information is implemented in the form of a specific illuminating of the rendering element, then the light intensity can be adapted according to the time that has elapsed since the date of creation so as to render the degree of obsolescence of the state information. In another variant given by way of an illustration of this characteristic, an increasing degree of obsolescence can be perceived by the reduction of an amplitude of shift if the rendering of the state information is implemented by an alternating motion of at least one part of the rendering element.

More generally, whatever the technique implemented to render the state information, it can be planned to act on a related rendering parameter available through this technique or to adapt this technique to take account of the obsolescence of this piece of information.

According to another particular aspect of an embodiment of the invention, the method may include a step for identifying persons belonging to a group comprising the following steps:
obtaining an identifier of said group;
searching within a database for an identity of each of the persons belonging to said group by means of said identifier.

Thus, it is possible for example to designate the group and the persons of the group by their pseudonyms the making it possible, if necessary, to maintain confidentiality and/or preserve the private lives of these individuals or secure the access to the rendering means.

According to yet another particular aspect of an embodiment of the invention, during the transforming step, said emotional state is converted into a control signal that can be interpreted by the emotion-rendering means.

Thus, the working and implementation of the rendering means are simplified and made reliable by the preliminary conversion of the emotional state into a control signal.

The method of an embodiment of the invention can especially comprise a step for rendering said emotional state information implementing at least one of the rendering forms belonging to the group comprising:
displaying still and/or moving images;
placing at least one moving part of said rendering element in a predetermined position;
placing at least one part of said rendering element in a predetermined state;
illuminating at least one part of said rendering element;
sound and/or voice rendering;
disseminating a piece of olfactory information;
tactile rendering;
taste rendering.

According to one particular characteristic of an embodiment of the invention, with the rendering step implementing at least one illumination of at least one part of a rendering element, the parameters for implementing said lighting belong to the group comprising:
color;
intensity;
pulsation.

Thus, it is possible to provide for a personalizing of the parameters for implementing the illumination of one or more rendering elements according to the preferences of the person using such elements.

According to another particular aspect of an embodiment of the invention, with said rendering means comprising means for the entry of at least one emotional state, said obtaining step comprises a step of entry of an emotional state by selection, using said entry means, of one state among a set of at least two predetermined states.

Thus, the person in the group can advantageously use the rendering means to declare his emotional state, for example by pressing specific buttons incorporated for this purpose into the rendering means.

An embodiment of the invention also relates to a system for transmitting information pertaining to at least two persons.

According to an embodiment of the invention, such an information transmitting system comprises:
means for obtaining emotional states associated with said persons;
means for transforming each emotional state into a piece of state information;
means for transmitting said state information to emotion-rendering means capable of associating, with each person, at least two rendering elements, each capable of taking at least two predetermined states representing at least two distinct respective emotional states.

It must be noted that, in certain complementary embodiments of the invention, such a system may include mean necessary for implementing one of the methods for transmitting information described here above.

An embodiment of the invention also pertains to a rendering terminal comprising means for rendering emotions capable of associating, with at least two persons, at least two respective rendering elements each capable of taking at least two predetermined states representing at least two distinct emotional states.

According to an embodiment of the invention, such a terminal comprises:
means for receiving at least two pieces of state information obtained by transformation of at least two associated emotional states;
means for selecting a state of at least two of said rendering elements as a function of said pieces of state information.

Thus, the means for selecting such a terminal can be used to make at least one rendering state correspond with each emotional state, for example by following pre-set rules or again according to a stochastic approach, and even when these states do not specifically coincide with the rendering states for which the rendering means are configured.

In one embodiment, said rendering means comprise a luminous frame.

Thus, if the frame is substantially the same as, for example, the rendering element, the person associated with the frame is quickly identified by the placing of a photograph or a picture of this person within the frame. Moreover, the user-friendly nature of this particular embodiment of the invention can be noted.

In another particular embodiment of the invention, said emotion-rendering means comprise a screen to represent the image of at least one avatar associated with one of said persons.

In one variant of the above embodiment, each avatar is constituted by a fish included in an aquarium and the state information associated therewith is rendered by means of at least one of the display parameters belonging to the group comprising:
color of said fish;
shape of the eye;
position of the fins;
emission of bubbles;
swimming speed;
amplitude of movement;
swimming depth.

Thus, a multimedia interface is exploited to make the rendering playful and enable intuitive perception, appreciably at a glance, of the emotional state of each person or more generally of the average emotional state of a group of persons with reference to the known natural behavior of living fish.

It is of course easy to imagine other types of depiction, for example in replacing the fish by animals, flowers or any other type of avatar.

According to one particular aspect of an embodiment of the invention, said emotional state is rendered according to a user's capabilities of perception. Thus, for example, for users who are enthusiastic about new technologies and/or enjoy interpreting complex information, the system may be designed so as to enable the refusal, as desired, of nuances in perceptible rendering states. On the contrary, in the case of use by young children or by persons who are reluctant to learn new techniques, or have impaired sensory faculties, it may be envisaged in one of the specific embodiments of the invention to adapt the rendering states so as to make them more easily accessible and prevent any ambiguity of interpretation. This may be implemented for example by reducing the number of emotional states that can be rendered by the rendering means.

An embodiment of the invention also relates to an information management server for managing information pertaining to at least two persons.

According to an embodiment of the invention, such a management server comprises:
means for obtaining an emotional state associated with each of said persons;
means for transforming each of said emotional states into a state information on emotions;
means for transmitting said state information on emotions to emotion-rendering means capable of associating, with each person, at least two rendering elements, each capable of taking at least two predetermined states representing at least two distinct respective emotional states.

Thus a management server of this kind enables a synchronizing of the information streams coming from different persons, especially when a time-out is planned for transmitting state information on emotions to the respective rendering means of these persons. Furthermore, in one particularly advantageous embodiment, such a server may include means for storing the emotional states of persons, for example in dynamic tables of a database.

An embodiment of the invention also concerns a computer program product downloadable from a communications network and/or stored on a computer-readable carrier and/or executable by a microprocessor, comprising program code instructions for the execution of all or part of the steps of at least one of the methods for transmitting information as described here above when it is executed on a computer.

### BRIEF DESCRIPTION OF THE DRAWINGS

Other features and advantages shall appear more clearly from the following description of two examples of preferred embodiments, given by way of simple, illustrative and non-exhaustive examples, and from the appended drawings, of which:

FIG. 1 illustrates a first example of an embodiment of a system for transmitting information according to an embodiment of the invention for which a rendering terminal of one of the persons takes the form of a set of photo frames;

FIG. 2 provides an illustration, in the form of a block diagram, of a succession of steps of the method according to an embodiment of the invention implemented before transmission of control information to the rendering terminal presented in FIG. 1;

FIG. 3 is a second example of an embodiment of the invention in which the persons of the group are represented on the screen in the form of fish swimming in an aquarium.

### DETAILED DESCRIPTION OF ILLUSTRATIVE EMBODIMENTS

An embodiment of the invention therefore proposes a novel approach that makes it possible to collect and transmit information on emotional states of several persons towards emotion-rendering means so as to be rendered therein in a form that can, in particular, be grasped simply and intuitively by a viewer.

Through an embodiment of the invention, each person, or member of a group, using unique rendering means, can thus access firstly the emotional state of the persons of the group that have declared a state and, secondly, an indication of the general emotional state of the group and can thus stay closer to the persons of the group and especially more alert and/or interactive with certain persons. An embodiment of the invention also enables each person of the group to share his emotions with this group.

In this context, it may be recalled that the implementation of an embodiment of the invention only requires that at least one person of a group should be connected to a communications network and that the other persons of the group should be able to link up to this communications network.

Besides, it will be seen more clearly here below in the description that the invention can be implemented without restriction by any type of rendering terminal connected to a communications network having available a number of rendering elements that is sufficient with respect to the number of persons in the group.

Thus, in one particular embodiment of the invention, the invention proposes to render the emotional states of all the persons, also called members, of a group on as many rendering elements as the group has persons.

Here below, by way of an example, we present the case of an implementation of the method according to an embodiment of the invention for transmitting pieces of information on emotional states to a rendering terminal comprising a set of frames illuminated from within.

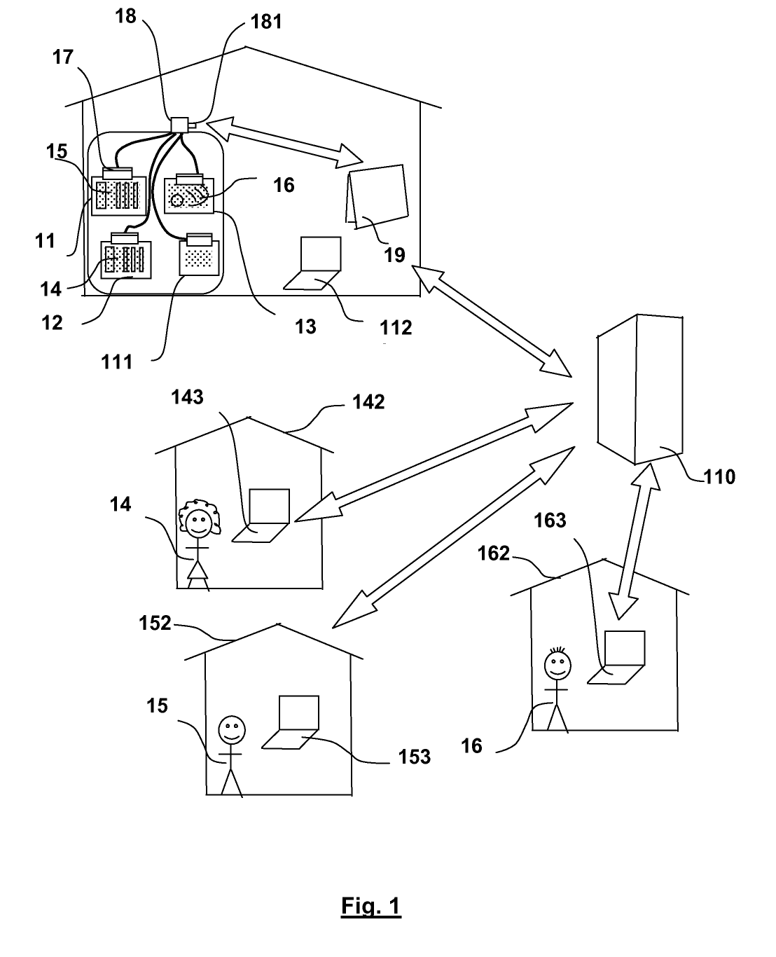

Thus, the user of the rendering terminal can personalize each frame by placing in it a photograph of a member of a group to which the frame has been allotted. In the implementation of this first example of an embodiment presented with reference to FIG. 1, the group to which the terminal has been assigned consists of members of the terminal user's family, among them his grandmother referenced 14, his father referenced 15 and his oldest child referenced 16.

It is clear however that the invention cannot be limited to this particular application but can also be implemented in many other fields, possibly leading to the emergence of uses that are as yet undetermined.

Thus, as can be seen in FIG. 1 which illustrates an example of a system for transmitting information according to an embodiment of the invention, the elements for rendering pieces of information on emotional states are frames 11, 12, 13 made of plexiglas illuminated from within by three trichromatic diodes, respectively colored blue, red and green. These frames can be fixed by magnetization, for example to the door of a fridge at the home of the rendering terminal user. Drilled holes (not shown) may also be planned in the thickness of the frame so as to enable the user to pin the frames to a wall. Photographs 141, 151 and 161 of the three members of the family mentioned here above, residing in the dwellings 142, 152 and 162, have been slid by the user respectively into the frames 11 to 13. They are held for example by means of clamps 17.

Each frame has an code wheel associated with a logic number ranging here from 1 to 4 and enabling the allocation of a frame to a particular member of the group.

Four-wire series type buses connect the frames 11 to 13 to a central control unit 18 which complements the rendering terminal. The unit 18 is connected to a module for communications with the wireless local area network 181, in this case through a WiFi (wireless fidelity) link with a home communications gateway 19 (for example a Livebox ®).

The control unit 18 connected to the network is configured for the transmission, at periodic intervals to a server 110 and through a gateway 19, of a request asking for transfer of control information corresponding to the pieces of information on emotional state given by the other members of the group and stored in the dynamic tables of a specific database hosted by the server 110.

After a check and a notification of permission to transfer, as the case may be, the pieces of information on emotional states are transmitted in return by the server. The form of rendering the rendering elements 11 to 13 concerned is then updated as a function of the control information transmitted to the unit through the gateway 19 by the server 110, for example according to the IP (Internet protocol) exchange protocol.

In other modes of implementation of the invention, the server could spontaneously carry out transfers of control information in order to execute an automatic updating of the rendering means of members who will have formulated preliminary requests, for example during a recording procedure with a manager of such a service.

In one particular embodiment of the invention, this rendering of the emotional state takes the form of a variation of the frame illumination colors. Seven rendering states corresponding to these colors may be obtained by powering one of the diodes or by powering a combination of two or three diodes.

It must be noted that, in this example of an embodiment of the invention, the information transmission system enables the user to parameterize the assigning of one of the seven frame illumination colors to the emotional states that can be rendered by his rendering terminal, by accessing for example through a computer 112, the hosted management application which manages the preferences of each user.

In variants of this embodiment, it can also be planned to act on the intensity of the illumination and/or the pulsation of the diodes to render an emotional state that can be interpreted by the user or the oldness (obsolescence) of the rendered state. In particular, the package has means to adjust the light intensity of the frame so as to dim it for example in proportion to the time elapsed since the date of creation of a stated emotional state.

It must furthermore be noted that if the emotional state of a member of the group cannot be obtained and/or converted, for example in the case of a break or a dysfunctioning of the network or again should the members not have communicated their emotional state for a long period, then a piece of state information of a neutral type, or information indicating an unavailability, is allotted to this member.

In one optional variant of this embodiment, a flashing of a frame at a specific frequency can be planned to inform the user that a member of the group with which this frame is associated has announced a change in his emotional state.

In this particular embodiment of the invention, the members 14, 15 and 16 forming part of the user's emotion-sharing group announce their emotional state by means of a specific application installed in their computer 143, 153, 163. This application can be used to display an entry window with a scrolling menu by which it is possible to obtain a scrolling of seven pictograms, also loosely called “emoticons”, representing the seven emotional states accessible in this embodiment and to select one of these pictograms to declare an emotional state at that same instant.

In one variant of this embodiment, it can be planned to select the emotional state that the members wish to declare by means of a specific interface, such as a rotating selector displayed in a window on the screen, or provided for this purpose, on the control unit.

According to another approach, a fourth frame 111 is associated with the user. It is provided on its rim with seven buttons, each corresponding to one of the seven emotional states (for example: tense, irritated, disillusioned, tired, curious, happy, neutral) accessible through the terminal in this particular embodiment. By simply pressing one of the buttons of the frame 111, the user can communicate his emotional state to the other members of this group.

Each frame can also be provided with a button which enables information to be sent on to the server, for example so that the user can inform the concerned member of the group that he has ascertained the emotional state and that he is getting ready to call him, especially if the emotional state declared by this member is the “tense” state.

This button, or a second button, can also be used to start a call directly. According to one aspect of an embodiment of the invention, means may be planned to automatically start a communication, for example upon reception of a predetermined piece of information on an emotional state.

In one variant of an embodiment of the invention, a switch is implemented in the frame to enable the user to use the rendering terminal in order to receive the emotional state of a second group grouping together friends of the user.

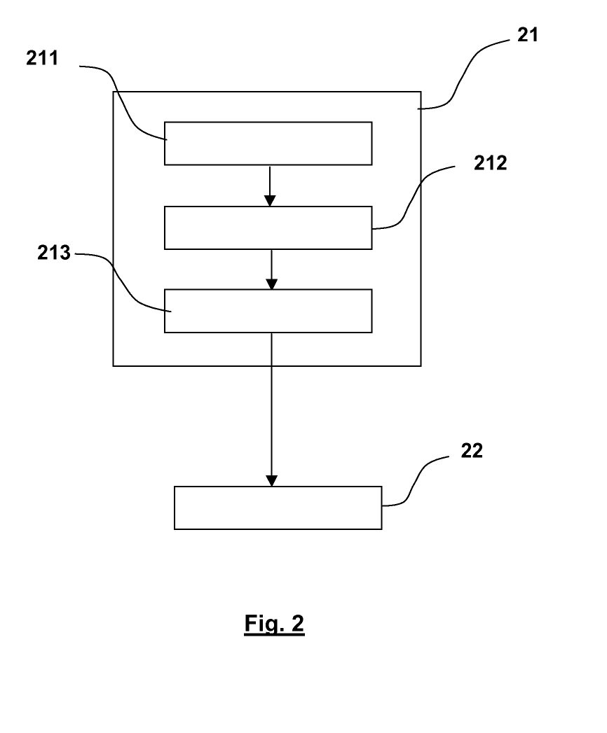

Referring to FIG. 2, we present the steps of the method according to an embodiment of the invention prior to the transmission of the pieces of control information to the user's rendering terminal, in the form of a block diagram.

In a first step 21, the pieces of information are obtained from the server 110.

In this embodiment, this obtaining step 21 comprises:
a first step 211 of identification by secured access, for example by a user name and a password, to the database of the server included in the request asking for transfer to the server;
a second step 212 for identifying the group (possibly designated by a pseudonym) for which the transfer is requested;
a third step 213 of searching for information on the group identified in the successive fields of the dynamic table assigned to the user for this group identified in the dedicated database of the server. Such fields include the designation of the group (if necessary by means of a pseudonym), the identity of the members of the group, the list of frames and members with which they have been associated, the emotional state declared by each member or assigned to each member by default, and as the case may be the date of creation of the emotional state.

The obtaining step 21 is followed by a step 22 for converting the emotional states obtained into pieces of information on emotional states taking the form of pieces of control information that can be interpreted by the rendering terminal. These pieces of control information in this first example of an embodiment of the invention take the form of a list of strings of characters encapsulated in a digital transmission file.

For facility of transmission of these encoding strings, it is possible for example to adopt a 16-bit encoding format. Thus, according to this format, the first bits can be allotted to the rendering of the color of the frame, the next two bits to the intensity of lighting and the last eight bits to the flashing commands for the flashing of the frame.

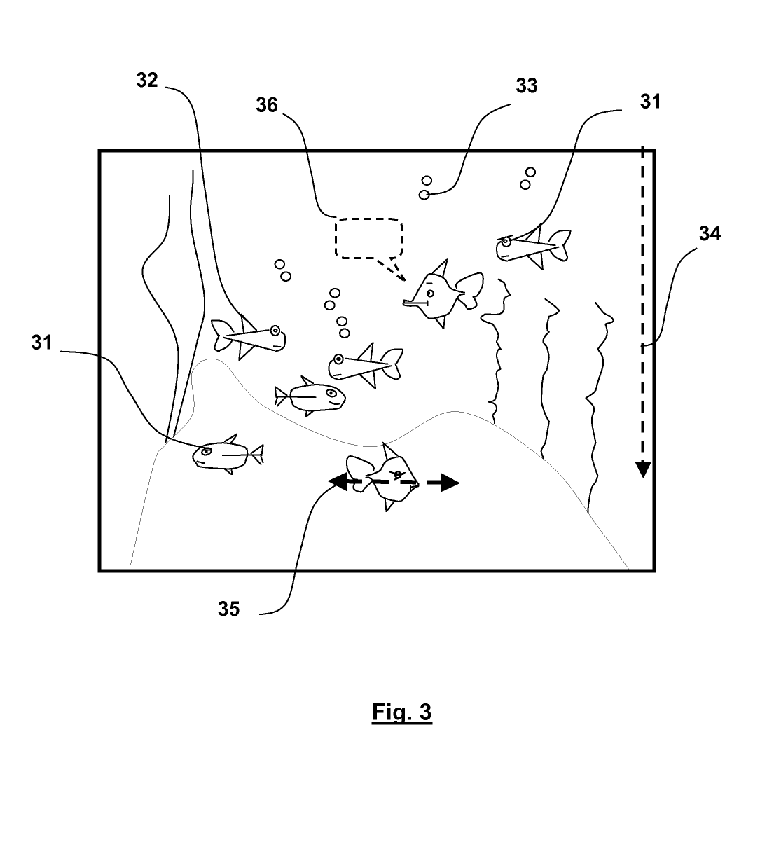

Referring now to FIG. 3, we present a second example of an embodiment of the invention in which the emotional states of the members of the group are rendered by the display of a graphic window (or a screen background) on the computer screen.

The user can thus consult the emotional states, transmitted by Internet, of the members of a predefined group using a dedicated application installed on his computer in viewing representations that are live or variable (at least depending on the emotional states) of the different members of the group. In this example described in this FIG. 3, these representations take the form of fish in an aquarium (defined by a graphic window).

Depending on parameters such as the appearance of the fish (color, the expression of the eye 31, the position of the fins 32, etc.), whether bubbles are being emitted or not, the swimming speed, the swimming depth 34, the amplitude of the movements 35 or again untimely changes in direction, the user can thus interpret the emotional state obtained and, as the case may be, declared by the members of the group.

It must be noted that, in this second embodiment of the invention, the amplitude of movement of the fish can be parameterized so as to be reduced as a function of the time that has elapsed since the date of creation of the emotional state driving the motion and the appearance of the fish. It can also be planned that the fish will move further away accordingly.

In this particular embodiment, the user can use a pointer to select a fish and cause the appearance of a window describing the member of the group associated with the fish. In one variant of this embodiment, it can also be planned that the selection of a fish will enable the opening of a “web” type Internet applications page to inform the user about the member associated with the fish or again to enter into contact, for example through the opening of a dialog window, with an instantaneous messaging service or through a new electronic mail using an electronic mail application.

A message written by a member of the group can also be rendered by “his” fish for example within a balloon 36 associated with him.

Furthermore, in one variant of this second embodiment of the invention, two extension modules for the rendering management application are planned to enable the user to draw the fish representing the members of the group, for example by providing an export interface based on an artistic creation application.

In alternative embodiments described in detail here above, it can be planned that:
the rendering elements comprise means, for example a screen or a speaker if it is a frame that is involved, to display or disseminate a message saved by a member of the group at the time of declaration of his emotional state on the server;
the terminal includes alerting means to report a change in state (for any change or for certain states and/or certain individuals identified) or to report that one's own emotional state has not been updated for a predetermined period of time;
the terminals used by the different members are all identical (but if necessary can be parameterized in different ways) or on the contrary are different. Indeed, an embodiment of the invention can be used to adapt information collected from the server to different terminals.

Although the present disclosure has been described with reference to one or more examples, workers skilled in the art will recognize that changes may be made in form and detail without departing from the scope of the disclosure and/or the appended claims.

1. A method for transmitting information pertaining to at least two persons, wherein the method includes at least the following steps:
obtaining emotional states associated with said persons;
transforming each of said emotional states into a piece of state information; and
transmitting said pieces of state information to emotion-rendering means capable of associating, with each person, at least two predetermined states representing at least two distinct respective emotional states.

2. The method for transmitting pieces of information according to claim 1, wherein the method comprises a step for putting at least one second person into communication with a first person who has preliminarily declared his emotional state.

3. The method for transmitting pieces of information according to claim 1, wherein the obtaining step comprises a step of declaration, by at least one of said persons, of his emotional state.

4. The method for transmitting pieces of information according to claim 3, wherein the declaring step comprises a step of associating a date of creation with said emotional state.

5. The method for transmitting pieces of information according to claim 4, wherein the step of transforming comprises a step of adapting the state information as a function of said date of creation.

6. The method for transmitting pieces of information according to any claim 1, wherein the method comprises a step of identifying persons belonging to a group comprising the following steps:
obtaining an identifier of said group;
searching within a database for an identity of each of the persons belonging to said group by said identifier.

7. The method for transmitting pieces of information according to claim 1 wherein, during the transforming step, said emotional state is converted into a control signal that can be interpreted by the emotion-rendering means.

8. The method for transmitting pieces of information according to claim 1, wherein the method comprises a step of rendering said emotional state information acting on at least one illumination of at least one part of a rendering element, in effecting variation in at least one of the parameters belonging to the group comprising:
color;
intensity;
pulsation.

9. The method for transmitting pieces of information according to any claim 1, wherein said rendering means comprising means for entry of at least one emotional state, said obtaining step comprises a step of entry of an emotional state by selection, using said entry means, of one state among a set of at least two predetermined states.

10. A system for transmitting information pertaining to at least two persons, wherein the system comprises:
means for obtaining emotional states associated with said persons;
means for transforming each emotional state into a piece of state information; and
means for transmitting said state information to emotion-rendering means capable of associating, with each person, at least two predetermined states representing at least two distinct respective emotional states.

11. A rendering terminal comprising:
means for rendering emotions capable of associating, with at least two persons, at least two respective rendering elements each capable of taking at least two predetermined states representing at least two distinct emotional states;
means for receiving at least two pieces of state information obtained by transformation of at least two associated emotional states; and
means for selecting a state of at least two of said rendering elements as a function of said pieces of state information.

12. The rendering terminal according to claim 11, wherein said rendering means comprise a luminous frame.

13. The rendering terminal according to claim 11, wherein said emotion-rendering means comprise a screen to represent the image of at least one avatar associated with one of said persons.

14. The rendering terminal according to claim 13, wherein each avatar is constituted by a fish included in an aquarium and the state information associated therewith is rendered by at least one of the display parameters belonging to the group comprising:
color of said fish;
shape of an eye of the fish;
position of fins of the fish;
emission of bubbles;
swimming speed;
amplitude of movement;
swimming depth.

15. The rendering terminal according to claim 11, wherein said emotional state is rendered according to a user's capabilities of perception.

16. A server for managing pieces of information pertaining to at least two persons, wherein the server comprises:
means for obtaining an emotional state associated with each of said persons;
means for transforming each of said emotional states into a state information on emotions; and
means for transmitting said state information on emotions to emotion-rendering means capable of associating, with each person, at least two predetermined states representing at least two distinct respective emotional states.

17. A computer program product stored on a computer-readable carrier, wherein the product comprises program code instructions for execution of all or part of the steps of of a method for transmitting information pertaining to at least two persons when the instructions are executed on a computer, wherein the method comprises:
obtaining emotional states associated with said persons;
transforming each of said emotional states into a piece of state information; and
transmitting said pieces of state information to emotion-rendering means capable of associating, with each person, at least two predetermined states representing at least two distinct respective emotional states.
